(19) United States
(12) Patent Application Publication
(10) Pub. No.: US 2021/0106240 A1
(43) Pub. Date: Apr. 15, 2021

(54) Fetal Heart Rate Prediction from Electrocardiogram

(71) Applicant: Owlet Baby Care, Inc., Lehi, UT (US)

(72) Inventors: Sean Kerman, Lehi, UT (US); Elliot Brown, Provo, UT (US); Tanner Christensen, Provo, UT (US); Chris Hettinger, Worcester, MA (US); Jeffrey Humpherys, Lehi, UT (US)

(21) Appl. No.: 17/065,454

(22) Filed: Oct. 7, 2020

Related U.S. Application Data
(60) Provisional application No. 62/939,829, filed on Nov. 25, 2019.
(60) Provisional application No. 62/911,889, filed on Oct. 7, 2019.

(51) Int. Cl. A61B 5/024; A61B 5/308; A61B 5/00; A61B 5/366

(52) U.S. Cl. CPC A61B 5/02411; A61B 5/308; A61B 5/7207; A61B 5/7267; A61B 5/366; A61B 5/7257

(57) ABSTRACT

A technology for obtaining a fetal heart rate from an electrocardiogram (ECG) signal. In one example, an artificial neural network model can be trained to predict a fetal heart rate using a training dataset containing ECG data. The artificial neural network model can include a first series of convolutional layers to separate a fetal ECG signal from a maternal ECG signal, a fast Fourier transform (FFT) layer to convert the fetal ECG signal to ECG frequency representations, and a dense layer to decode the ECG frequency representations to fetal heart rate predictions. After training the artificial neural network model, ECG data generated by an ECG monitor can be obtained, and the ECG data can be input to the artificial neural network model. The artificial neural network model outputs a fetal heart rate prediction, wherein the fetal heart rate prediction represents the fetal heart rate obtained from the ECG signal.

### PRIORITY CLAIM

This application claims priority to Application Ser. No. 62/911,889 filed on Oct. 7, 2019 entitled FETAL HEART RATE ESTIMATION FROM ELECTROCARDIOGRAM and Application Ser. No. 62/939,829 filed on Nov. 25, 2019 entitled FETAL HEART RATE PREDICTION FROM ELECTROCARDIOGRAM. Both of the foregoing applications are incorporated herein by reference in their entireties.

### BACKGROUND

Non-invasive health monitoring devices are increasingly helping people to better monitor their health status both at an activity/fitness level for self-health tracking and at a medical level providing more data to clinicians with a potential for earlier diagnostic and guidance of treatment. Some consumer wearable devices have incorporated pulse-oximetry as a sensor for gathering biometric data. While pulse-oximetry data can provide several important insights into an individual's health, there is a desire for increased monitoring that provides information beyond what a standard pulse-oximeter is able to provide. For example, standard wrist-worn pulse-oximeters are unable to provide health information for a fetus of a pregnant woman. One potential means for providing fetal health information is through the use of abdominal electrodes for fetal monitoring. Abdominal electrodes for fetal monitoring are non-invasive and can potentially allow for in-home and ambulatory use.

### BRIEF DESCRIPTION OF THE DRAWINGS

FIG. 1 is a block diagram illustrating an example of a processing system used to generate a fetal heart rate probability.

FIG. 2 is a flow diagram that illustrates an example method for preprocessing ECG data.

FIG. 3 is a block diagram illustrating an example processing system which can incorporate prior heart rate predictions to determine a fetal heart rate probability.

FIG. 4 is a block diagram illustrating an example network architecture for an artificial neural network model which incorporates prior heart rate information to generate a fetal heart rate probability.

FIG. 5 is a block diagram that illustrates an example artificial neural network model configured to generate peak probabilities that correspond to individual heart rate peaks which can be decoded to produce a final fetal heart rate prediction.

FIG. 6 is a block diagram illustrating an example artificial neural network model trained using prior fetal heart rate predictions.

FIG. 7 is a flow diagram that illustrates an example method for training an artificial neural network model to predict a fetal heart rate from an ECG signal.

FIG. 8 is a flow diagram that illustrates an example method for obtaining a fetal heart rate prediction from an artificial neural network model.

FIG. 9 is block diagram illustrating an example of a computing device that may be used to execute a method for obtaining a fetal heart rate prediction from an artificial neural network model.

FIG. 10 is an illustration of an end-to-end artificial neural network architecture configured to generate fetal heart rate probabilities based on ECG data input.

FIG. 11 is an illustration of an end-to-end artificial neural network architecture configured to generate incorporate prior heart rate information into an artificial neural network model to generate a fetal heart rate probability.

FIG. 12 is an illustration of an artificial neural network architecture configured to generate a peak probability.

FIG. 13 is an illustration of an ECG passing prior prediction information to neural network model.

FIG. 14 is an illustration showing the original ECG signal.

FIG. 15 is an illustration showing the preprocessed ECG signal

FIG. 16 is an illustration that shows the ECG signal and a Fourier transform of the ECG signal after passing through the convolutional layers of the artificial neural network model described in association with FIG. 1

FIG. 17 is also an illustration that shows the ECG signal and a Fourier transform of the ECG signal after passing through the convolutional layers of the artificial neural network model described in association with FIG. 1.

FIG. 18 is an illustration showing the final heart rate probability output of the neural network model.

### DETAILED DESCRIPTION

Technologies are described for extracting a fetal heart rate from an electrocardiogram (ECG) signal acquired from an array of electrodes on a maternal abdomen using an artificial neural network and a Fourier transform. In one example, an artificial neural network model can be trained to predict a fetal heart rate using a training dataset containing ECG data. The artificial neural network model can include a first series of convolutional layers used to identify a fetal heart rate from a maternal heart rate, a fast Fourier transform (FFT) layer used to identify ECG frequencies in the ECG data, and a dense layer (also referred to as a dense decoding layer) used to decode a heart rate value from the ECG frequencies. After training the artificial neural network model, an ECG signal can be obtained from a fetal heart rate monitor and ECG data representing the ECG signal can be input to the artificial neural network model, and the artificial neural network model outputs a fetal heart rate prediction that represents a fetal heart rate extracted from the ECG signal.

In one example, the artificial neural network model can include an additional step that removes maternal artifacts from the ECG signal. For example, a maternal heart rate represented in the ECG signal can sometimes produce a spurious fetal heart rate. This maternal heart rate that resembles a fetal heart rate in the ECG signal can be incorrectly classified as a fetal heart rate. As such, in one example, a maternal signal cancellation step can be performed after the first series of convolutional layers and before the FFT layer to remove the maternal signal from the ECG data. In another example, the maternal signal cancellation step can be performed before the first series of convolutional layers. As will be appreciated, the artificial neural network model can be configured to perform maternal signal cancellation at any time to remove maternal artifacts from the ECG signal.

The network architecture of the artificial neural network model described herein provides improvements in the accuracy of fetal heart rate predictions obtained from an ECG signal over previous methods for computing a fetal heart rate from an ECG signal. In particular, the accuracy of fetal heart rate predictions output by the artificial neural network model is improved by placing an FFT layer after a first series of convolutional layers and providing the output of the FFT layer to a dense layer of the artificial neural network model. Placement of the FFT layer in this way improves the accuracy of fetal heart rate predictions by using the FFT layer to identify fundamental and harmonic frequencies of a fetal ECG signal, thereby reducing a number of parameters that are provided to the dense layer of the artificial neural network model.

Previous methods for computing a fetal heart rate from abdominal electrodes identify R-peaks of a maternal ECG in an ECG signal and then create a template of the maternal ECG on each channel. The template of the maternal ECG is subtracted out of the ECG signal leaving a residual ECG signal containing the fetal ECG. Following the maternal ECG cancellation, some methods have used a source separation algorithm to attempt to group the fetal ECG signal into fewer channels. Following source separation, a peak detection method has been used to identity fetal R-peaks on each channel. After fetal R-peaks are identified, a channel selection algorithm has been used to choose the best peaks from each channel to produce the final R-peak locations. These R-peak locations have been used to compute the fetal heart rate heart rate. With the present technology described herein, there is no need to build a maternal template to cancel out a maternal ECG signal. There is also no need for source separation algorithms or a need to individually process channels.

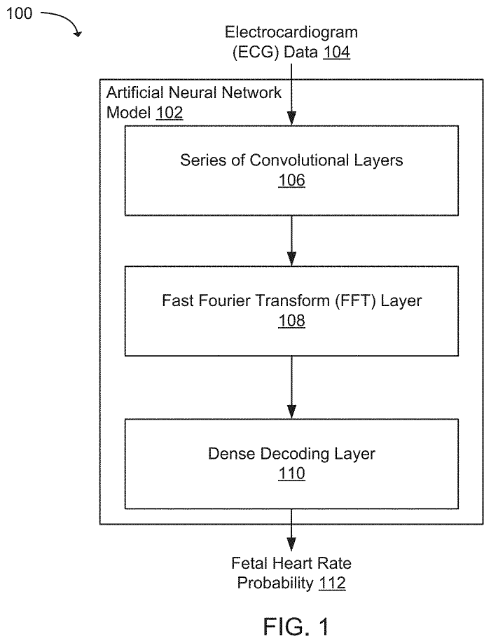

To further describe the present technology, examples are now provided with reference to the figures. FIG. 1 is a block diagram illustrating a high-level example of a processing system 100 used to generate a fetal heart rate probability. The processing system 100 includes an artificial neural network model 102 configured to extract a fetal heart rate from an electrocardiogram (ECG) signal. The artificial neural network model 102 (also referred to as the neural network model 102), in one example, is an end-to-end neural network having an architecture that includes a series of convolutional layers 106 followed by a fast Fourier transform (FFT) layer 108 and a dense decoding layer 110. As described in more detail below, ECG data 104 can be provided as input to the neural network model 102 and the architecture of the neural network model 102 can be configured to analyze the ECG data 104 to identify a fetal ECG signal and process the fetal ECG signal to generate a fetal heart rate probability 112.

As illustrated, the architecture of the neural network model 102 includes a series of convolutional layers 106. The function of the convolutional layers 106 can include amplifying and reshaping QRS maternal and QRS fetal complexes while filtering out activity in an ECG signal. The series of convolutional layers 106 can include any number of convolutional layers. In a specific example of the architecture of the neural network model 102, the series of convolutional layers 106 can include three convolutional layers. The convolutional layers 106 can be configured to separate a maternal ECG signal from a fetal ECG signal. An ECG signal obtained from the maternal abdomen of a pregnant subject includes ECG information for both the subject and the subject's fetus. As an illustration, electrodes can be attached to the maternal abdomen of a pregnant subject and the electrical signals of the subject's and the fetus' hearts can be recorded. Consequently, the ECG signal obtained from the maternal abdomen of the pregnant subject includes artifacts of a maternal ECG signal. The convolutional layers 106 of the neural network model 102 can be configured to remove the maternal ECG signal artifacts (as well as other artifacts) from the ECG signal to better isolate a fetal ECG signal.

In some examples, the series of convolutional layers 106 may be a first convolutional layer that proceeds the FFT layer 108, and the architecture of the neural network model 102 can include a second series of convolutional layers (not shown) located between the FFT layer 108 and the dense decoding layer 110. The second series of convolutional layers is similar to the first but acts on the frequency transformed signal allowing the signal to be cleaned in the frequency domain potentially improving signal quality. For example, the second series of convolutional layers can be used to identify and remove artifacts from a Fourier transform output by the FFT layer 108, which may further improve a fetal ECG signal.

The architecture of the neural network model 102 shown in FIG. 1 places the FFT layer 108 between the convolutional layer 106 and the dense decoding layer 110. The FFT layer 108 can be configured to apply a fast Fourier transform technique to a fetal ECG signal output by the series of convolutional layers 106 to convert the fetal ECG signal to a representation of a fundamental frequency and harmonic frequencies. Applying a fast Fourier transform technique to a fetal ECG signal allows resulting fetal ECG frequencies to be quantized into values that can be classified into heart rate values.

Placing the FFT layer 108 after the series of convolutional layers 106 and before the dense decoding layer 110 improves performance of predicting fetal heart rates using the neural network model 102. In particular, applying a fast Fourier transform technique to a fetal ECG signal output by the series of convolutional layers 106 reduces a number of parameters that are provided to the dense decoding layer 110 of the neural network model 102. By reducing the number of parameters provided as input to the dense decoding layer 110, an amount of data processed by the dense decoding layer 110 is decreased, which results in a shorter amount of time to generate fetal heart rate probabilities 112.

Also, placing the FFT layer 108 after the series of convolutional layers 106 and before the dense decoding layer 110 improves accuracy of fetal heart rate predictions output by the neural network model 102. More specifically, applying a fast Fourier transform technique to fetal ECG signals output by the series of convolutional layers 106 allows fetal ECG frequencies to be quantized, thereby restricting a number of possible fetal ECG frequency values that can be classified as heart rate values. This allows for the use of classification, as opposed to using regression, which can produce bias in the ECG data. As an example, using a means squared error technique as a loss function tends to pull values toward the mean, which creates bias in the ECG data. Using a fast Fourier transform technique reduces the chance of bias in the ECG data. For example, a fast Fourier transform technique allows fetal ECG frequencies output by the FFT layer 108 to be classified as a probability distribution of fetal heart rates, and allows for a maximum likelihood estimation to be applied to the probability distribution of fetal heart rates to determine a fetal heart rate probability 112.

Accuracy of fetal heart rate predictions can be further improved by removing spurious fetal heart rate values from the ECG data. For example, a maternal heart rate represented in an ECG signal can sometimes produce a spurious fetal heart rate. This maternal heart rate can resemble a fetal heart rate in the ECG signal and can be incorrectly classified as a fetal heart rate. This spurious fetal heart rate can be removed from the ECG data using an additional step that removes maternal artifacts from the ECG signal. The additional step can identify the spurious fetal heart rate produced by the maternal ECG signal that resembles a fetal heart rate and remove the spurious fetal heart rate from the ECG dataset. This step, in one example, can be performed after the first series of convolutional layers and before the FFT layer, or in another example, before the first series of convolutional layers. The architecture of the neural network model 102 can include a maternal signal cancellation technique that removes spurious fetal heart rate. The maternal signal cancellation technique can include two parts. The first part determines whether a fetal heart rate is close in time to a maternal heart rate (e.g., within 0.02 seconds, 0.05 seconds, 0.07 seconds, etc. of the maternal heart rate). If the fetal heart rate is close in time to the maternal heart rate, then the fetal heart rate may be ignored. The second part inserts zero or null values in the region of the fetal FFT that corresponds to the maternal heart rate to further ensure that the maternal heart rate is not accidentally reported as the fetal heart rate.

The dense decoding layer 110 included in the neural network model 102 architecture can be configured to decode ECG frequency representations output by the FFT layer 108 into fetal heart rate predictions. In one example, the dense decoding layer 110 decodes the ECG frequency representations into fetal heart rate information (e.g., Beats Per Minute (BPM)) used to generate a fetal heart rate prediction. As an example, the dense decoding layer 110 selects an ECG frequency representation (e.g., a harmonic frequency) output by the FFT layer 108 and applies a mask to the ECG frequency representation which is used to visualize the ECG frequency representation as a fetal heart rate value (e.g., 110, 130, or 160 BPM). Thereafter, the fetal heart rate values can be scored to create a probability distribution that indicates a maximum likelihood of a fetal heart rate, which can be output as a fetal heart rate probability 112. In one example, after scoring the fetal heart rate values, the fetal heart rate values can be input to a softmax layer (shown in Example 1) that has one neuron for each fetal heart rate value. The softmax layer can normalize the fetal heart rate values to sum to a value of 1, creating a probability distribution of fetal heart rate values that indicates a maximum likelihood of a fetal heart rate value.

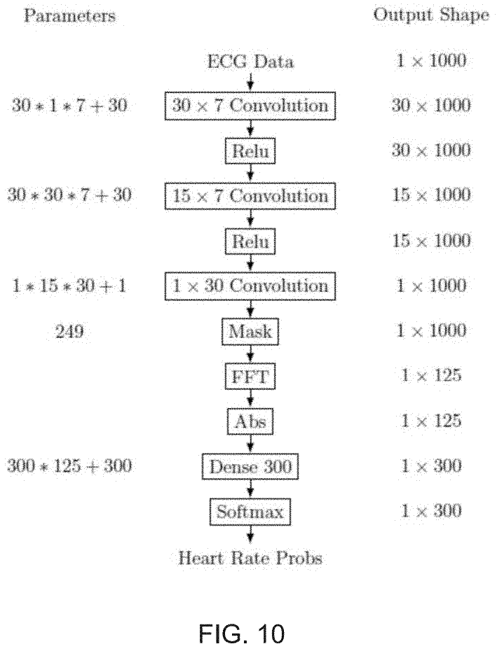

As will be appreciated, the example artificial neural network architecture shown in FIG. 10 is merely representative of a neural network architecture and is not meant to be limiting.

The neural network model 102 can be trained to generate fetal heart rate probabilities using a training dataset of ECG data. The training dataset can comprise ECG data collected from a number of pregnant subjects using an ECG monitor. The ECG data can be obtained using ECG electrodes collected, for example, at 1 kHz. In one example, an ultrasound monitor can be used to acquire a fetal heart rate reference from one or more pregnant subjects. The ECG data can be low pass filtered and down sampled (e.g., to 200 Hz). The ECG data can then be divided into a training dataset and a test dataset. In one example, the neural network model 102 can be trained using categorical cross entropy to label ECG data in the training dataset to a heart rate category (e.g., number of fetal heart beats per minute categories) and an Adam optimizer to update weights assigned to the ECG data. In another example, the neural network model 102 can be trained using binary cross entropy, mean squared error, least absolute deviation, or another appropriate loss function. Once the peak probabilities have been generated, they can be decoded into peak locations and fetal heart rate using a variety of known peak detection methods. As will be appreciated, techniques other than those described above can be used to train the neural network model 102.

Fetal heart rate probabilities 112 (also referred to as fetal heart rate predictions) output by the neural network model 102 can be stored to a storage medium (e.g., RAM, hard-drive, and the like) included in a computing device (e.g., a wearable device) in order to make the fetal heart rate probabilities 112 available in response to requests for the fetal heart rate probabilities 112 (e.g., a request from an application or cloud service). In one example, a computing device that hosts the neural network model 102 can send fetal heart rate probabilities 112 over a network (e.g., Internet, intranet, WAN, etc.) to another device (e.g., a mobile device, a server, etc.) in response to a request for the fetal heart rate probabilities 112. Also, it is contemplated that a fetal heart rate probability 112 can be displayed on a display of a wearable device.

The various processes and/or other functionality of the processing system 100 may be executed on one or more processors that are in communication with one or more memory modules. The processing system 100 may include one or more computing devices. In some examples, the processing system 100 can include a data store used to store ECG data 104 and/or fetal heart rate probabilities 112 output by the neural network model 102. The term “data store” may refer to any device or combination of devices capable of storing, accessing, organizing and/or retrieving data. The storage system components of the data store may include storage systems such as volatile or non-volatile RAM, hard-drive type media, and a cloud storage network. The data store may be representative of a plurality of data stores as can be appreciated.

In some examples, the processing system 100 may include a network for transmitting data between servers, clients, and devices. The network may include any useful computing network, including an intranet, the Internet, a local area network, a wide area network, a wireless data network, or any other such network or combination thereof. Components utilized for such a system may depend at least in part upon the type of network and/or environment selected. Communication over the network may be enabled by wired or wireless connections and combinations thereof.

FIG. 1 illustrates that certain processing modules may be discussed in connection with this technology and these processing modules may be implemented as computing services. In one example configuration, a module may be considered a service with one or more processes executing on a server or other computer hardware. Such services may be centrally hosted functionality or a service application that may receive requests and provide output to other services or consumer devices. For example, modules providing services may be considered on-demand computing that are hosted in a server, virtualized service environment, grid or cluster computing system. An API may be provided for each module to enable a second module to send requests to and receive output from the first module. Such APIs may also allow third parties to interface with the module and make requests and receive output from the modules. While FIG. 1 illustrates an example of a system that may implement the techniques above, many other similar or different environments are possible. The example environments discussed and illustrated above are merely representative and not limiting.

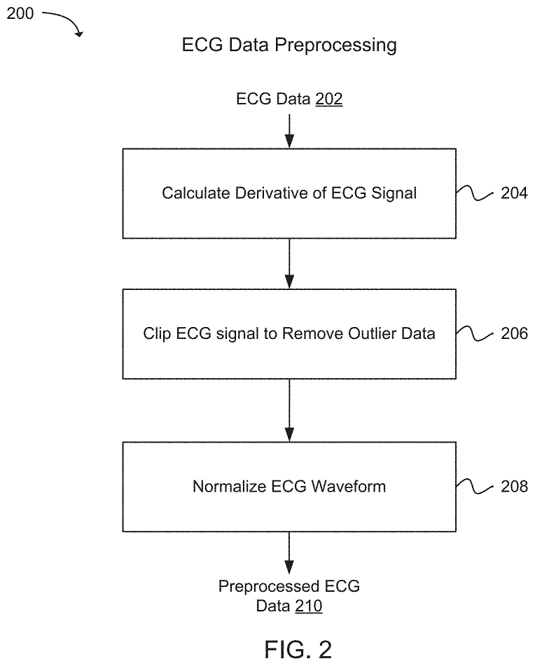

FIG. 2 is a flow diagram that illustrates an example method 200 for preprocessing ECG data 202. Prior to inputting ECG data 202 to the neural network model 102 described above, the ECG data 202 can be preprocessed to format the ECG data 202 for input to the neural network model 102. Preprocessing can be performed prior to training the neural network model 102, and prior to inference time in which a predicted fetal heart rate value is generated using the trained neural network model. Also, it is contemplated that ECG data 202 generated by an ECG sensor included in a wearable device (e.g., a bellyband fetal monitoring device) can be preprocessed prior to inputting the ECG data 202 to the neural network model 102.

Preprocessing of ECG data 202 can include one or more preprocessing steps. In one example, the preprocessing steps can include: (i) calculating a derivative of an ECG signal to accentuate high frequency components in the ECG signal, (ii) clipping the ECG signal to remove outlier data included in the ECG data, and (iii) normalizing an ECG waveform of the ECG signal to a predetermined standard deviation.

As in block 204, the preprocessing step of taking a derivative of an ECG signal included in ECG data 202 can be performed to accentuate high frequency components of a fetal QRS complex (combination of three of the graphical deflections seen on a typical electrocardiogram) in the ECG signal. This step can be performed to emphasize the QRS complex in the ECG data so that the information is more apparent when processed by the neural network model 102.

As in block 206, the preprocessing step of clipping an ECG signal included in ECG data 202 can be performed to remove outlier data included in the ECG data 202. Outliers can be caused by either the maternal QRS complex or maternal movement. In one example, clipping an ECG signal can include computing amplitude percentiles of the ECG signal and clipping values that are greater than a difference between amplitude percentiles. As an illustration, the 25th, 50th, and 75th amplitude percentiles of an ECG signal can be computed, and values can be clipped that are greater than six (6) times the difference between the 50th and 75th percentile of less than six (6) times the difference between the 50th percentile and the 25th percentile.

As in block 208, the preprocessing step of normalizing an ECG waveform of an ECG signal to a predetermined standard deviation can be performed to ensure that ECG signals included in ECG data 202 are approximately the same scale. After preprocessing ECG data, the preprocessed ECG data 210 can be provided as input to the neural network model 102 to generate fetal heart rate probabilities as described earlier.

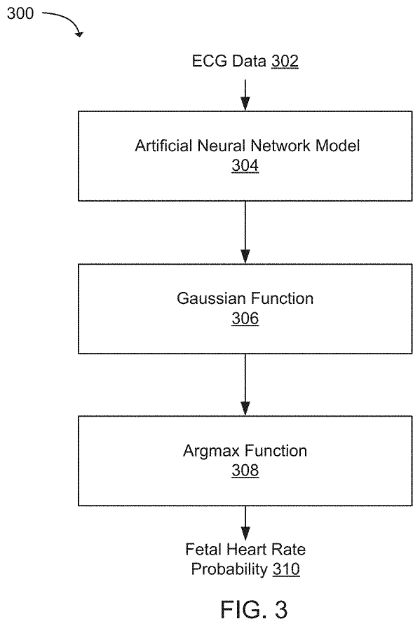

FIG. 3 is a block diagram illustrating an example of a processing system 300 which can incorporate prior heart rate predictions to determine a fetal heart rate probability 310. In particular, the processing system 300 can incorporate prior predictions of fetal heart rates into current predictions of fetal heart rates at inference time. The prior predictions can be used to gauge whether a current prediction is reasonable. For example, a sudden change in a fetal heart rate is rare. Therefore, a prior heart rate prediction can be compared to a current heart rate prediction to determine whether a change in heart rate from the prior prediction to the current prediction is a data anomaly or is reasonable. Accordingly, a current heart rate prediction that is close to a previous heart rate prediction can be treated as being more likely accurate than current heart rate predictions that are far away from previous heart rate predictions.

In the example illustrated in FIG. 3, the processing system 300 can include an artificial neural network model 304. The artificial neural network model 304 can have the architecture described earlier in association with FIG. 1. In addition to the artificial neural network model 304, the processing system 300 can include a Gaussian function 306 and an argmax function 308. One or more prior fetal heart rate predictions can be incorporated into a current fetal heart rate prediction at inference time (i.e., at the time the trained neural network model 304 is used to generate a fetal heart rate probability) by multiplying a probability distribution of fetal heart rates output by the artificial neural network model 304 by the Gaussian function 306. In one example, the Gaussian function 306 can have a mean value that is equal to the previous heart rate prediction. As an illustration, a fetal heart rate distribution can be multiplied by the Gaussian function 306 with a standard deviation of ten (10) BPM and a mean value equal the most recent heart rate prediction. In one example, if no recent heart rate prediction is available, the Gaussian function 306 can be replaced with an identity vector. The identity vector can be a vector of one (1) values of the same length as the Gaussian vector, resulting in not modifying the input to the argmax function 308 described below. In another example where no recent heart rate prediction is available, the Gaussian function 306 step described above may not be performed, and the argmax function 308 described below can be applied to the probability distribution . . . .

After multiplying the probability distribution by the Gaussian function 306, an argmax function 308 (arguments of the maxima) can be used to produce the fetal heart rate probability 310. For example, an argmax of the heart rate distribution can be calculated to produce the fetal heart rate probability 310. The probability of the heart rate value can be used as a quality indicator for fetal heart rate values. Fetal heart rates with low probability may be rejected to avoid false heart rate readings.

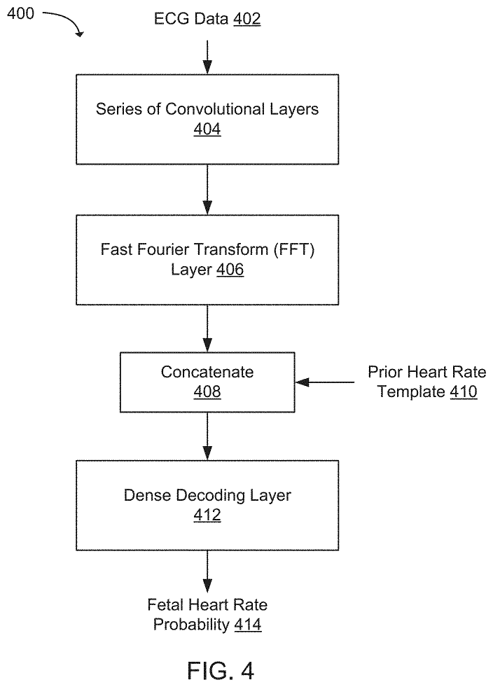

FIG. 4 is a block diagram that illustrates an example network architecture for an artificial neural network model 400 that incorporates prior heart rate information to generate a fetal heart rate probability 414. In the discussion of the processing system 300 above (shown in FIG. 3), prior heart rate information is incorporated after generating a probability distribution of fetal heart rates. The network architecture of the artificial neural network model 400 incorporates prior heart rate information into the neural network model 400 to allow training of the neural network model 400 to include the prior heart rate information.

A prior prediction of a fetal heart rate can be used as part of generating a current fetal heart rate probability in a number of ways. In one example, a series of sine waves corresponding to a fundamental frequency and harmonic frequencies of a prior fetal heart rate prediction can be summed. The resulting sum provides a prior heart rate template 410 that can be passed to the neural network model 400. One method that can be used to pass a prior heart rate template 410 to the neural network model 400 includes taking a Fourier transform of the prior heart rate template 410 and concatenating the Fourier transform to the Fourier transform output by the FFT layer 406 of the neural network model 400.

As an illustration, ECG data 402 included in a training dataset can be input to a series of convolutional layers 404 to separate a fetal ECG signal from a maternal ECG signal. The FFT layer 406 can be applied to the fetal ECG signal to produce a Fourier transform of the fetal ECG signal. A Fourier transform of a prior heart rate template 410 can be produced, and the Fourier transform of a prior heart rate template 410 can be concatenated 408 to the Fourier transform of the fetal ECG signal. The resulting concatenated Fourier transform comprising ECG frequency representations of the ECG data 402 and the prior heart rate template 410 can be input to a dense decoding layer 412 of the neural network model 400. The dense decoding layer 412 decodes the ECG frequency representations, as described earlier in association with FIG. 1, and outputs a fetal heart rate probability 414.

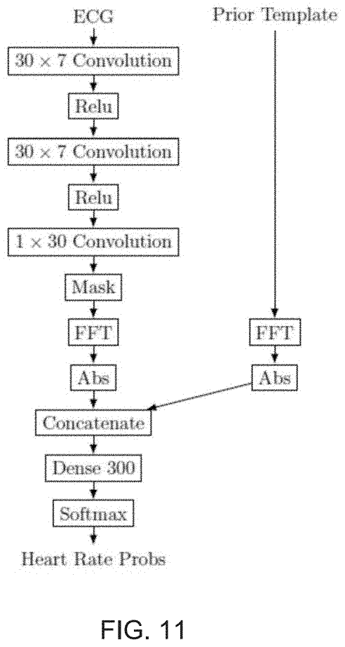

The example artificial neural network architecture shown in FIG. 11 is merely representative of a neural network architecture and is not limiting.

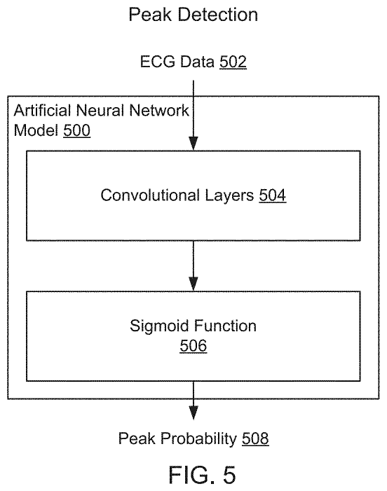

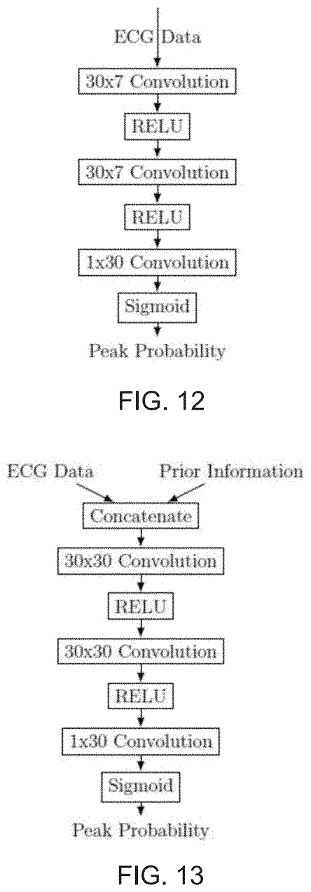

FIG. 5 is a block diagram that illustrates an example artificial neural network model 500 configured to generate peak probabilities 508 that correspond to individual heart rate peaks which can be decoded to produce a final fetal heart rate prediction. For example, peak probabilities 508 can be decoded using peak detection to produce a final heart rate probability. In one example, a peak detection method can use the preprocessing technique described above in association with FIG. 2 to preprocess ECG data 502. Following preprocessing, a waveform can be passed to the neural network model 500 in short windows (e.g., 100, 125, or 150 samples). The neural network model 500 can include of a series of convolutional layers 504 with a sigmoid function 506 output corresponding to a peak probability 508. As will be appreciated, the example artificial neural network architecture shown in FIG. 12 is merely representative and is not meant to be limiting.

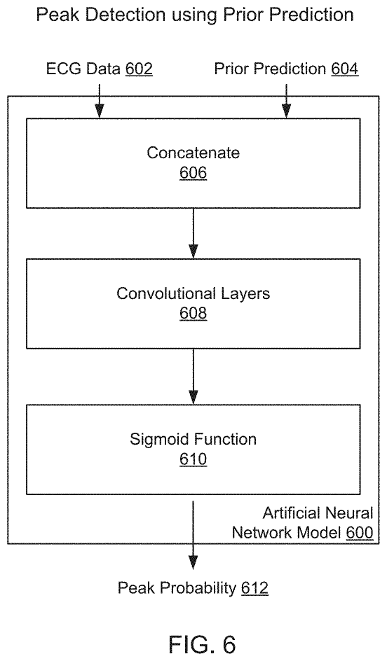

FIG. 6 is a block diagram illustrating an example artificial neural network model 600 trained using prior fetal heart rate predictions. The neural network model 600 can be trained using binary cross entropy, mean squared error, least absolute deviation, or another appropriate loss function. The neural network model 600 can include a concatenate layer 606, a series of convolutional layers 608, and a sigmoid function 640 layer. After peak probabilities have been generated, the peak probabilities can be decoded into peak locations and fetal heart rate using a variety of known peak detection methods. The neural network model 600 can be provided with information from prior fetal prior heart rate predictions (e.g., a prior prediction 604 input). For example, the expected location of the next peak can be passed to the neural network model 600 in addition to ECG data 602. One example of passing prior predictions 604 to the neural network model 600 includes adding an additional input channel that is the same length as the ECG signal, where the input channel has a value of one (1) when the next fetal heart rate is expected to be zero (0). The neural network model 600 can learn to incorporate the prior prediction 604 to improve prediction accuracy. As described earlier, the peak probabilities can be decoded into peak locations and a fetal heart rate using a variety of known peak detection methods.

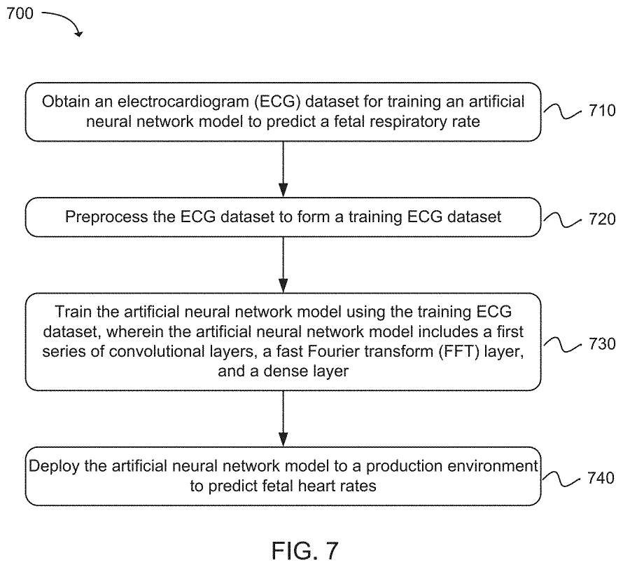

FIG. 7 is a flow diagram illustrating an example method 700 for training an artificial neural network model to generate a fetal heart rate prediction from an ECG signal. As in block 710, an ECG dataset can be obtained to train the artificial neural network model to predict a fetal heart rate. Prior to training the artificial neural network model using the ECG dataset, the ECG dataset can be preprocessed, as in block 720. Preprocessing the ECG dataset can include (i) calculating a derivative of the ECG signal to accentuate high frequency components of a fetal QRS complex in the ECG dataset, (ii) clipping the ECG signal to remove outlier data included in the ECG dataset, and (iii) normalizing an ECG waveform of the ECG signal to a predetermined standard deviation.

As in block 730, the artificial neural network model can be trained using the training ECG dataset. The artificial neural network model can include a first series of convolutional layers to separate a fetal ECG signal from a maternal ECG signal, a fast Fourier transform (FFT) layer to convert the fetal ECG signal to ECG frequency representations, and a dense layer to decode the ECG frequency representations to fetal heart rate predictions. In one example, an output layer of the neural network model is a softmax layer that has an output neuron for each heart rate value. In one example, the artificial neural network model can be trained using categorical cross entropy to label the ECG data in the training dataset to a heart rate category and an Adam optimizer to update weights assigned to the ECG data. In some examples, prior heart rate information can be used to train the artificial neural network model. For example, a prior heart rate template can be generated by summing a series of sine waves that correspond to a fundamental frequency of a prior heart rate prediction and a harmonic of the prior heart rate prediction, and the prior heart rate template can be input to the artificial neural network model during training of the artificial neural network model. Inputting a prior heart rate prediction to the artificial neural network model during training can include (i) calculating a Fourier transform of a prior fetal heart rate prediction, (ii) concatenating the Fourier transform of the prior fetal heart rate prediction to a Fourier transform output by the FFT layer to form a concatenated Fourier transform, and (iii) providing the concatenated Fourier transform to the dense layer of the artificial neural network model.

After training the artificial neural network model, the artificial neural network model can be deployed to a production environment (e.g., a wearable device, a mobile device, a server, or the like) to predict fetal heart rates, as in block 740. As a specific example, the artificial neural network model can be installed on a bellyband fetal monitoring device containing one or more sensors that detect electrical signals of a pregnant subject's heart and a fetus' heart. After deploying the artificial neural network to a production environment, ECG data generated by an ECG sensor or monitor can be input to the artificial neural network model, and the artificial neural network model can analyze the ECG data to determine a fetal heart rate prediction.

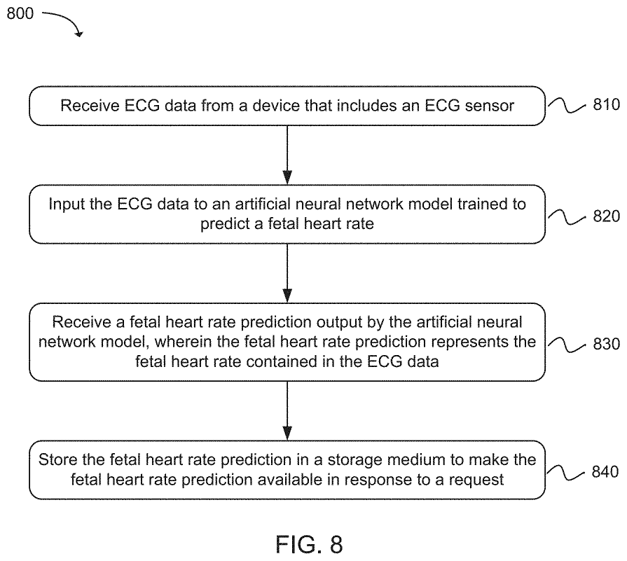

FIG. 8 is a flow diagram that illustrates an example method 800 for obtaining a fetal heart rate prediction from an electrocardiogram. As in block 810, ECG data can be received from a device that includes an ECG sensor, such as a wearable device. The device may be one of many physical electronic devices that create a large network of addressable devices. The devices may be part of a “network” that is commonly referred to as the Internet of Things (IoT). The devices that make up the network may be addressable over a wireless network, such as WI-FI, Zigbee, Z-Wave, BLUETOOTH, NFC (Near Field Communication), cellular, and the like.

In one example, the ECG data received from the device can be preprocessed prior to inputting the ECG data to the artificial neural network. For example, preprocessing the ECG data can include one or more of: calculating a derivative of the ECG signal to accentuate high frequency components of a fetal QRS complex in the ECG data, clipping the ECG signal to remove outlier data included in the ECG data, and/or normalizing an ECG waveform of the ECG signal to a predetermined standard deviation.

As in block 820, the ECG data can be input to an artificial neural network model trained to predict a fetal heart rate. As described earlier, the artificial neural network model can include: a first series of convolutional layers to separate a fetal ECG signal from a maternal ECG signal, a fast Fourier transform (FFT) layer to convert the fetal ECG signal to ECG frequency representations, and a dense layer to decode the ECG frequency representations to fetal heart rate predictions. In one example, an output layer of the neural network model is a softmax layer that has an output neuron for each heart rate value. In one example, a previous heart rate prediction output by the artificial neural network model can be obtained, and the previous heart rate prediction can be compared to a current heart rate prediction output by the artificial neural network model to determine whether the current heart rate prediction is within a quality threshold of the previous heart rate prediction. For example, the previous heart rate prediction can be compared to the current heart rate prediction by (i) generating a heart rate distribution, wherein the current heart rate prediction is multiplied by a Gaussian function that has a mean value that is equal to the previous heart rate prediction, and (ii) calculating an argmax of the heart rate distribution to produce the heart rate prediction. In the case that a previous heart rate prediction is unavailable, a heart rate distribution can be generated by multiplying a current heart rate prediction by an identity vector and calculating an argmax of the heart rate distribution to produce the heart rate prediction.

As in block 830, output of a fetal heart rate prediction can be received from the artificial neural network model. The fetal heart rate prediction can represent the fetal heart rate obtained from the ECG signal. In one example, the heart rate prediction can be evaluated based on previous heart rate predictions output by the artificial neural network model to determine a probability that the heart rate prediction is accurate, and the heart rate prediction can be discarded when the probability is low that the heart rate prediction is accurate.

As in block 840, the fetal heart rate prediction can be stored in a storage medium (e.g., RAM, hard-drive, flash memory, etc.) to make the fetal heart rate prediction available in response to a request from an application, program, service, process, and the like. It is contemplated that the fetal heart rate prediction output by the artificial neural network model can be sent over a network to another device, such as a mobile device or server, in response to a request for the fetal heart rate prediction. In one example, the fetal heart rate prediction can be pushed to another device, such as a mobile device or server, using a push protocol. For example, the device may be configured to communicate with other computing devices using either TCP (Transmission Control Protocol) or UDP (User Datagram Protocol) protocols. Likewise, the device may be programmed to communicate with other computing devices using any suitable protocol including, for example, MQTT (Message Queue Telemetry Transport), CoAP (Constrained Application Protocol), HTTP (Hypertext Transfer Protocol), and HTTPS (HTTP secure). For example, some devices may transport data using TCP, while others may communicate data using UDP. Some devices may use MQTT, while others may use CoAP, and still others may use HTTPs. It is also contemplated that the fetal heart rate prediction can be displayed on the display of a wearable device that hosts the artificial neural network model.

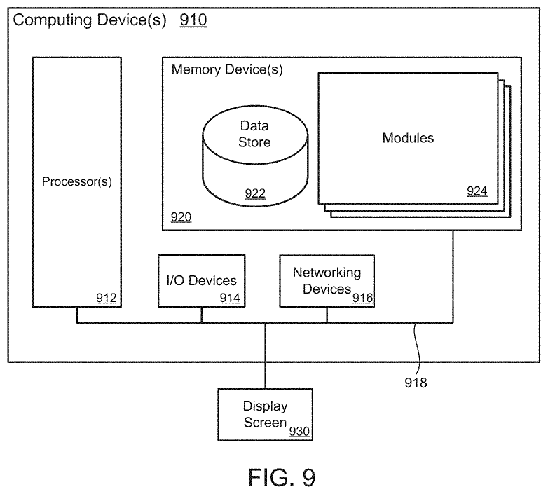

FIG. 9 illustrates a computing device 910 on which modules of this technology may execute. A computing device 910 is illustrated on which a high-level example of the technology may be executed. The computing device 910 may include one or more processors 912 that are in communication with memory devices 920. The computing device 910 may include a local communication interface 918 for the components in the computing device 910. For example, the local communication interface 918 may be a local data bus and/or any related address or control busses as may be desired.

The memory device 920 may contain modules 924 that are executable by the processor(s) 912 and data for the modules 924. The modules 924 can include convolutional modules, fast Fourier transform modules, dense decoding modules, and other modules. The modules 924 may execute the functions described earlier. A data store 922 may also be located in the memory device 920 for storing data related to the modules 924 and other applications along with an operating system that is executable by the processor(s) 912.

Other applications may also be stored in the memory device 920 and may be executable by the processor(s) 912. Components or modules discussed in this description that may be implemented in the form of software using high-level programming languages that are compiled, interpreted or executed using a hybrid of the methods.

The computing device 910 may also have access to I/O (input/output) devices 914 that are usable by the computing device 910. One example of an I/O device is a display screen 930 that is accessible to the computing device 910. Networking devices 916 and similar communication devices may be included in the computing device 910. The networking devices 916 may be wired or wireless networking devices that connect to the interne, a LAN, WAN, or other computing network.

The components or modules that are shown as being stored in the memory device 920 may be executed by the processor(s) 912. The term “executable” may mean a program file that is in a form that may be executed by a processor 912. For example, a program in a higher level language may be compiled into machine code in a format that may be loaded into a random access portion of the memory device 920 and executed by the processor 912, or source code may be loaded by another executable program and interpreted to generate instructions in a random access portion of the memory to be executed by a processor. The executable program may be stored in any portion or component of the memory device 920. For example, the memory device 920 may be random access memory (RAM), read only memory (ROM), flash memory, a solid state drive, memory card, a hard drive, optical disk, floppy disk, magnetic tape, or any other memory components.

The processor 912 may represent multiple processors and the memory device 920 may represent multiple memory units that operate in parallel to the processing circuits. This may provide parallel processing channels for the processes and data in the system. The local communication interface 918 may be used as a network to facilitate communication between any of the multiple processors and multiple memories. The local communication interface 918 may use additional systems designed for coordinating communication such as load balancing, bulk data transfer and similar systems.

While the flowcharts presented for this technology may imply a specific order of execution, the order of execution may differ from what is illustrated. For example, the order of two more blocks may be rearranged relative to the order shown. Further, two or more blocks shown in succession may be executed in parallel or with partial parallelization. In some configurations, one or more blocks shown in the flow chart may be omitted or skipped. Any number of counters, state variables, warning semaphores, or messages might be added to the logical flow for purposes of enhanced utility, accounting, performance, measurement, troubleshooting or for similar reasons.

Some of the functional units described in this specification have been labeled as modules, in order to more particularly emphasize their implementation independence. For example, a module may be implemented as a hardware circuit comprising custom VLSI circuits or gate arrays, off-the-shelf semiconductors such as logic chips, transistors, or other discrete components. A module may also be implemented in programmable hardware devices such as field programmable gate arrays, programmable array logic, programmable logic devices or the like.

Modules may also be implemented in software for execution by various types of processors. An identified module of executable code may, for instance, comprise one or more blocks of computer instructions, which may be organized as an object, procedure, or function. Nevertheless, the executables of an identified module need not be physically located together, but may comprise disparate instructions stored in different locations which comprise the module and achieve the stated purpose for the module when joined logically together.

Indeed, a module of executable code may be a single instruction, or many instructions and may even be distributed over several different code segments, among different programs and across several memory devices. Similarly, operational data may be identified and illustrated herein within modules and may be embodied in any suitable form and organized within any suitable type of data structure. The operational data may be collected as a single data set, or may be distributed over different locations including over different storage devices. The modules may be passive or active, including agents operable to perform desired functions.

The technology described here may also be stored on a computer readable storage medium that includes volatile and non-volatile, removable and non-removable media implemented with any technology for the storage of information such as computer readable instructions, data structures, program modules, or other data. Computer readable storage media include, but is not limited to, a non-transitory machine readable storage medium, such as RAM, ROM, EEPROM, flash memory or other memory technology, CD-ROM, digital versatile disks (DVD) or other optical storage, magnetic cassettes, magnetic tapes, magnetic disk storage or other magnetic storage devices, or any other computer storage medium which may be used to store the desired information and described technology.

The devices described herein may also contain communication connections or networking apparatus and networking connections that allow the devices to communicate with other devices. Communication connections are an example of communication media. Communication media typically embodies computer readable instructions, data structures, program modules and other data in a modulated data signal such as a carrier wave or other transport mechanism and includes any information delivery media. A “modulated data signal” means a signal that has one or more of its characteristics set or changed in such a manner as to encode information in the signal. By way of example and not limitation, communication media includes wired media such as a wired network or direct-wired connection and wireless media such as acoustic, radio frequency, infrared and other wireless media. The term computer readable media as used herein includes communication media.

Reference was made to the examples illustrated in the drawings and specific language was used herein to describe the same. It will nevertheless be understood that no limitation of the scope of the technology is thereby intended. Alterations and further modifications of the features illustrated herein and additional applications of the examples as illustrated herein are to be considered within the scope of the description.

Furthermore, the described features, structures, or characteristics may be combined in any suitable manner in one or more examples. In the preceding description, numerous specific details were provided, such as examples of various configurations to provide a thorough understanding of examples of the described technology. It will be recognized, however, that the technology may be practiced without one or more of the specific details, or with other methods, components, devices, etc. In other instances, well-known structures or operations are not shown or described in detail to avoid obscuring aspects of the technology.

Although the subject matter has been described in language specific to structural features and/or operations, it is to be understood that the subject matter defined in the appended claims is not necessarily limited to the specific features and operations described above. Rather, the specific features and acts described above are disclosed as example forms of implementing the claims. Numerous modifications and alternative arrangements may be devised without departing from the spirit and scope of the described technology.

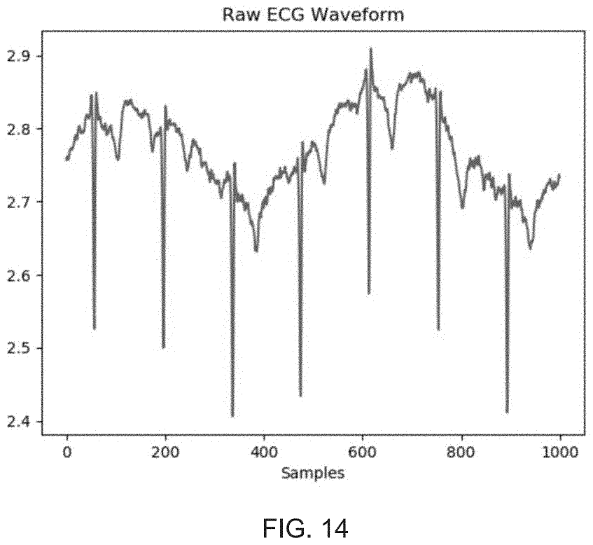

The following examples illustrate what an ECG signal looks like at various stages in the processing chain. For the sake of simplicity, the examples show a single channel of ECG as other channels typically look similar. FIG. 14 shows the original ECG signal. The dominant peaks in the ECG signal represent the maternal ECG signal. The fetal ECG is not visible on the waveform.

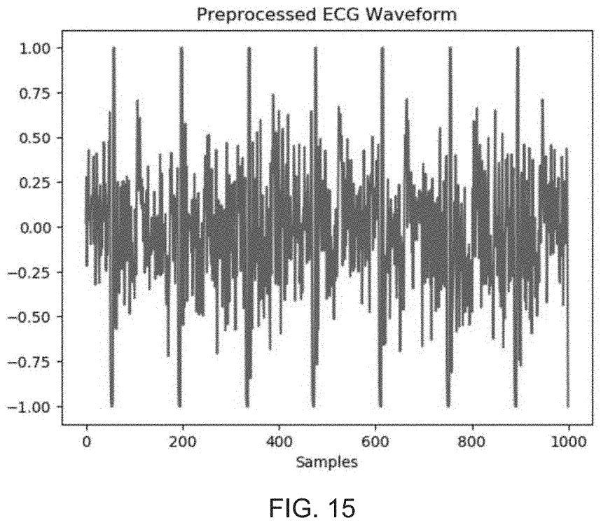

FIG. 15 shows the preprocessed ECG signal. The fetal ECG in the ECG signal is difficult to identify.

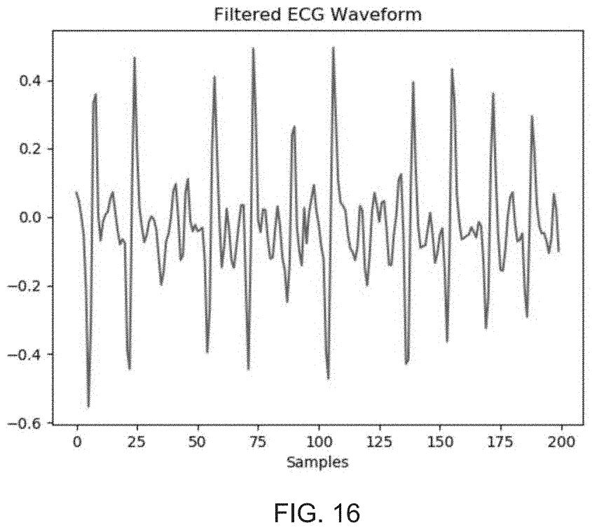

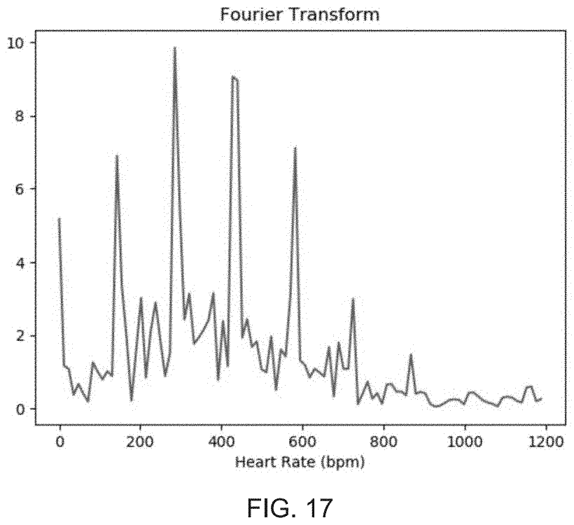

FIGS. 16 and 17 show the ECG signal and a Fourier transform of the ECG signal after passing through the convolutional layers of the artificial neural network model described in association with FIG. 1.

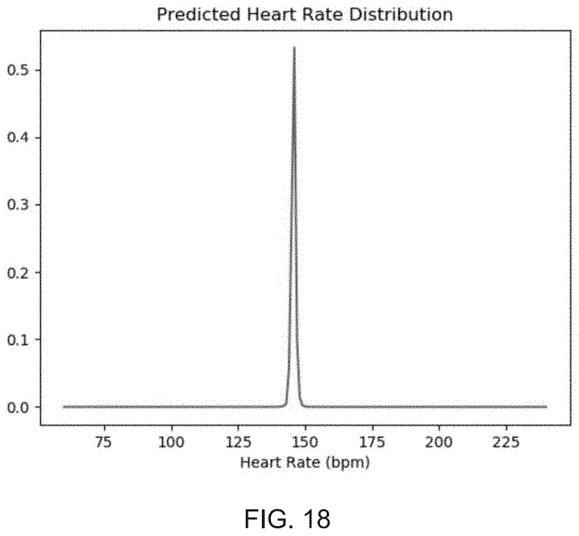

As shown in FIG. 17, the ECG data is substantially cleaner as compared to the ECG data shown in preceding FIGs., and the Fourier transform has a clear peak at the fetal heart rate value (approximately 145 BPM). The other large peaks are the harmonics of the fundamental heart rate frequency. This Fourier transform can be decoded by the dense decoding layer of the artificial neural network model. The output of the dense decoding layer is shown in FIG. 18 showing that there is a clear peak in probability at the correct heart rate value.

What is claimed is:

1. A system for predicting a fetal heart rate from an electrocardiogram (ECG) signal, comprising:
at least one processor;
a memory device including instructions that, when executed by the at least one processor, cause the system to:
input ECG data to an artificial neural network model trained to predict a fetal heart rate using the ECG data,
wherein the artificial neural network model includes a first series of convolutional layers to separate a fetal ECG signal from a maternal ECG signal, a fast Fourier transform (FFT) layer to convert the fetal ECG signal to ECG frequency representations, and a dense layer to decode the ECG frequency representations to fetal heart rate predictions;
receive a fetal heart rate prediction output by the artificial neural network model; and
provide the fetal heart rate prediction.

2. The system in claim 1, wherein the artificial neural network model is trained using categorical cross entropy to label the ECG data in a training dataset to a heart rate category and an Adam optimizer to update weights assigned to the ECG data.

3. The system in claim 1, wherein the memory device further includes instructions that, when executed by the at least one processor, cause the system to preprocess the ECG data , wherein preprocessing includes (i) calculating a derivative of the ECG signal to accentuate high frequency components of a fetal QRS complex in the ECG data, (ii) clipping the ECG signal to remove outlier data included in the ECG data, and (iii) normalizing an ECG waveform of the ECG signal to a predetermined standard deviation.

4. The system in claim 1, wherein the memory device further includes instructions that, when executed by the at least one processor, cause the system to:
generate a prior fetal heart rate template by summing a series of sine waves that correspond to a fundamental frequency of a prior fetal heart rate prediction and a harmonic of the prior fetal heart rate prediction; and
input the prior fetal heart rate template to the artificial neural network model during training of the artificial neural network model.

5. The system in claim 4, wherein inputting the prior fetal heart rate prediction to the artificial neural network model during training further comprises:
calculating a Fourier transform of the prior fetal heart rate prediction;
concatenating the Fourier transform of the prior fetal heart rate prediction to a Fourier transform output by the FFT layer to form a concatenated Fourier transform; and
providing the concatenated Fourier transform to the dense layer of the artificial neural network model.

6. The system in claim 1, wherein the first series of convolutional layers comprises three convolutional layers.

7. The system in claim 1, wherein an output layer of the neural network model is a softmax layer that has an output neuron for each fetal heart rate value.

8. The system in claim 1, wherein the neural network model further includes a second series of convolutional layers between the FFT layer and the dense layer to remove artifacts from a Fourier transform output by the FFT layer.

9. The system in claim 1, wherein the memory device further includes instructions that, when executed by the at least one processor, cause the system to:
obtain a previous fetal heart rate prediction output by the artificial neural network model; and
compare the previous fetal heart rate prediction to a current fetal heart rate prediction output by the artificial neural network model to determine whether the current fetal heart rate prediction is within a quality threshold of the previous fetal heart rate prediction.

10. The system in claim 9, wherein the previous fetal heart rate prediction is compared to the current fetal heart rate prediction by:
generating a heart rate distribution, wherein the current fetal heart rate prediction is multiplied by a Gaussian function that has a mean value that is equal to the previous fetal heart rate prediction; and
calculating an argmax of the heart rate distribution to produce the fetal heart rate prediction.

11. The system in claim 1, wherein the memory device further includes instructions that, when executed by the at least one processor, cause the system to:
determine that a previous fetal heart rate prediction output by the artificial neural network model is unavailable;
generate a heart rate distribution by multiplying a current fetal heart rate prediction by an identity vector; and
calculating an argmax of the heart rate distribution to produce the fetal heart rate prediction.

12. The system in claim 1, wherein the memory device further includes instructions that, when executed by the at least one processor, cause the system to:
evaluate the fetal heart rate prediction based on previous fetal heart rate predictions output by the artificial neural network model to determine a probability that the fetal heart rate prediction is accurate; and
discard the fetal heart rate prediction when the probability is low that the fetal heart rate prediction is accurate.

13. A computer implemented method obtaining fetal heart rate predictions from electrocardiograms, comprising:
receiving electrocardiogram (ECG) data from a device that includes an ECG sensor;
inputting the ECG data to an artificial neural network model trained to predict a fetal heart rate,
wherein the artificial neural network model includes a first series of convolutional layers to separate a fetal ECG signal from a maternal ECG signal, a fast Fourier transform (FFT) layer to convert the fetal ECG signal to ECG frequency representations, and a dense layer to decode the ECG frequency representations to fetal heart rate predictions;
receiving a fetal heart rate prediction output by the artificial neural network model, wherein the fetal heart rate prediction represents the fetal heart rate contained in the ECG data; and
storing the fetal heart rate prediction in a storage medium to make the fetal heart rate prediction available in response to a request.

14. The computer implemented method in claim 13, further comprising preprocessing the ECG data received from the device prior to inputting the ECG data to the artificial neural network.

15. The computer implemented method in claim 13, further comprising comparing a previous fetal heart rate prediction to a current fetal heart rate prediction to determine whether the current fetal heart rate prediction is within a quality threshold of the previous fetal heart rate prediction.

16. The computer implemented method in claim 13, further comprising sending the fetal heart rate prediction over a network to another device in response to a request for the fetal heart rate prediction.

17. The computer implemented method in claim 13, further comprising displaying the fetal heart rate prediction on a display included in the device.

18. A non-transitory machine readable storage medium including instructions embodied thereon, wherein the instructions, when executed by at least one processor:
obtain an electrocardiogram (ECG) dataset for training an artificial neural network model to predict a fetal heart rate;
preprocess the ECG dataset to form a training ECG dataset;
train the artificial neural network model using the training ECG dataset, wherein the artificial neural network model includes a first series of convolutional layers to separate a fetal ECG signal from a maternal ECG signal, a fast Fourier transform (FFT) layer to convert the fetal ECG signal to ECG frequency representations, and a dense layer to decode the ECG frequency representations to fetal heart rate predictions; and
deploy the artificial neural network model to a production environment to predict fetal heart rates.

19. The non-transitory machine readable storage medium in claim 18, further comprising instructions, that when executed by the at least one processor;
calculate a Fourier transform of a prior fetal heart rate prediction;
concatenate the Fourier transform of the prior fetal heart rate prediction to a Fourier transform output by the FFT layer to form a concatenated Fourier transform; and
provide the concatenated Fourier transform to the dense layer of the artificial neural network model.

20. The non-transitory machine readable storage medium in claim 18, wherein the instructions, that when executed by at least one processor, train the artificial neural network model, further train the artificial neural network model using (i) categorical cross entropy to label the ECG dataset as a fetal heart rate category and (ii) an Adam optimizer to update weights assigned to the ECG data.

21. The non-transitory machine readable storage medium in claim 18, further comprising instructions, that when executed by the at least one processor:
identify a spurious fetal heart rate produced by a maternal ECG signal that resembles a fetal heart rate; and
remove the spurious fetal heart rate from the ECG dataset.
